(19) United States
(12) Patent Application Publication
(10) Pub. No.: US 2014/0048079 A1
(43) Pub. Date: Feb. 20, 2014

(54) DISINFECTING MOUTH GUARD FOR VAP PREVENTION

(71) Applicant: Excelsior Medical Corporation, Neptune, NJ (US)

(72) Inventors: Christopher E. Gardner, Manalapan, NJ (US); William Anderson, Cary, IL (US)

(73) Assignee: Excelsior Medical Corporation, Neptune, NJ (US)

(21) Appl. No.: 13/968,151

(22) Filed: Aug. 15, 2013

Related U.S. Application Data
(60) Provisional application No. 61/683,658, filed on Aug. 15, 2012.

(51) Int. Cl. A61M 16/04; A61M 1/00; A61C 5/14

(52) U.S. Cl. CPC A61M 16/0497; A61C 5/14; A61M 1/0023
USPC 128/860; 128/861; 128/859

(57) ABSTRACT

The mouth guard includes first and second side walls, first and second curved walls, and a central wall having a top and a bottom surface. The curved walls are connected to the sidewalls, such that they define a periphery of the mouth guard. The central wall extends between the first and second sidewalls, and the first and second curved walls. The mouth guard includes top and bottom channels for receiving a patient's upper and lower teeth, respectively. First and second protrusions extend from the top surface of the central wall, and a first tube channel is formed between the first and second protrusions. A second tube channel is formed in the central wall and is connected with the first tube channel. An opening is formed in the curved walls and is in communication with the second tube channel. The first and second channels, and the opening receive an intubation tube.

### RELATED APPLICATIONS

The present application claims the priority of U.S. Provisional Application Ser. No. 61/683,658 filed Aug. 15, 2012, the disclosure of which is expressly incorporated herein by reference in its entirety.

### BACKGROUND

1. Field of the Disclosure

The present disclosure relates generally to disinfecting mouth guards to prevent ventilator associated pneumonia (VAP).

2. Related Art

Ventilator Associated Pneumonia (VAP) is one of the major categories of Healthcare Acquired Infections (HAI) in hospitals today. It is the second most common HAI, and the most common HAI in the ICU. 86% of nosocomial pneumonias are associated with mechanical ventilation. Koenig, Steven M. et al, “Ventilator-Associated Pneumonia: Diagnosis, treatment, and prevention,” Clinical Microbiology Review, October 2006, 19(4): 637-657. Between 8 and 28% of patients receiving mechanical ventilation are affected by VAP. Mortality for VAP ranges from 24-50% and can reach 76% in some specific settings or when lung infection is caused by high risk pathogens. Chastre J, et al. “Ventilator-associated Pneumonia,” American Journal of Respiratory and Critical Care Medicine, Apr. 1, 2002, 165(7): 867-903. In the United States alone, between 250,000 and 300,000 cases of VAP occur per year. Koenig, Steven M. et al, “Ventilator-Associated Pneumonia: Diagnosis, treatment, and prevention,” Clinical Microbiology Review, October 2006, 19(4): 637-657. VAP occurs more often in the first few days after intubation. VAP has been associated with increased intensive care unit costs and increased intensive care unit length of stays, which can be from 4-13 days. Additionally, incremental costs associated with VAP have been estimated as being between $5,000-$20,000 per diagnosis. Koenig, Steven M. et al, “Ventilator-Associated Pneumonia: Diagnosis, treatment, and prevention,” Clinical Microbiology Review, October 2006, 19(4): 637-657.

Recent studies have shown a significant improvement in patient outcome when a threefold approach is taken for those patients on ventilators. This threefold approach includes, keeping the patient elevated at a 30-45 degree angle, challenging the patient daily to determine if they still need breathing support, and daily oral hygiene. The oral hygiene is typically provided by nurses by performing an oral scrub with sponge tipped oral swabs that are soaked in a 0.12% chlorhexidine gluconate solution every 6-8 hours.

### SUMMARY

The present disclosure relates to a disinfecting mouth guard for VAP prevention. The mouth guard includes first and second side walls, first and second curved walls, and a central wall having a top and a bottom surface. The first and second curved walls are connected to the first and second sidewalls. The side walls and the curved walls define a periphery of the mouth guard. The central wall extends between the first and second sidewalls, and the first and second curved walls. The mouth guard includes top and bottom channels for receiving a patient's upper and lower teeth, respectively. First and second protrusions extend from the top surface of the central wall, and a first tube channel is formed between the first and second protrusions. A second tube channel is formed in the central wall and is connected with the first tube channel. An opening is formed in the curved walls and is in communication with the second tube channel. The first and second channels, and the opening receive an intubation tube.

In another embodiment, the mouth guard comprises a body shaped to cover at least a portion of a patient's mouth and an antimicrobial substance. The antimicrobial substance prevents infections associated with ventilation of a patient.

In still another embodiment, the mouth guard comprises a strip, a means for adhering the strip to a patient's mouth, and an antimicrobial substance suitable for preventing infections associated with ventilation of a patient. The strip includes the antimicrobial substance. The means for adhering the strip can be an adhesive.

The present disclosure further relates to a method of preventing ventilator associated pneumonia. The method comprises providing a mouth guard having an antimicrobial substance. The mouth guard is positioned in a patient's mouth, and an intubation tube is inserted into the patient's mouth. The antimicrobial substance disinfects at least a portion of the patient's mouth to prevent infection.

### BRIEF DESCRIPTION OF THE DRAWINGS

The foregoing features of the disclosure will be apparent from the following Detailed Description, taken in connection with the accompanying drawings, in which:

FIG. 1 is a top perspective view of the mouth guard;

FIG. 2 is a bottom perspective of the mouth guard;

FIG. 3 is a right side view of the mouth guard;

FIG. 4 is a rear view of the mouth guard;

FIG. 5 is a top view of the mouth guard;

FIG. 6 is a front view of another embodiment of the mouth guard, which includes a level indicating device;

FIG. 7 is a front view of still another embodiment of the mouth guard, formed as an adhesive strip; and

FIG. 8 is a perspective view showing the mouth guard of FIG. 7 in use.

### DETAILED DESCRIPTION

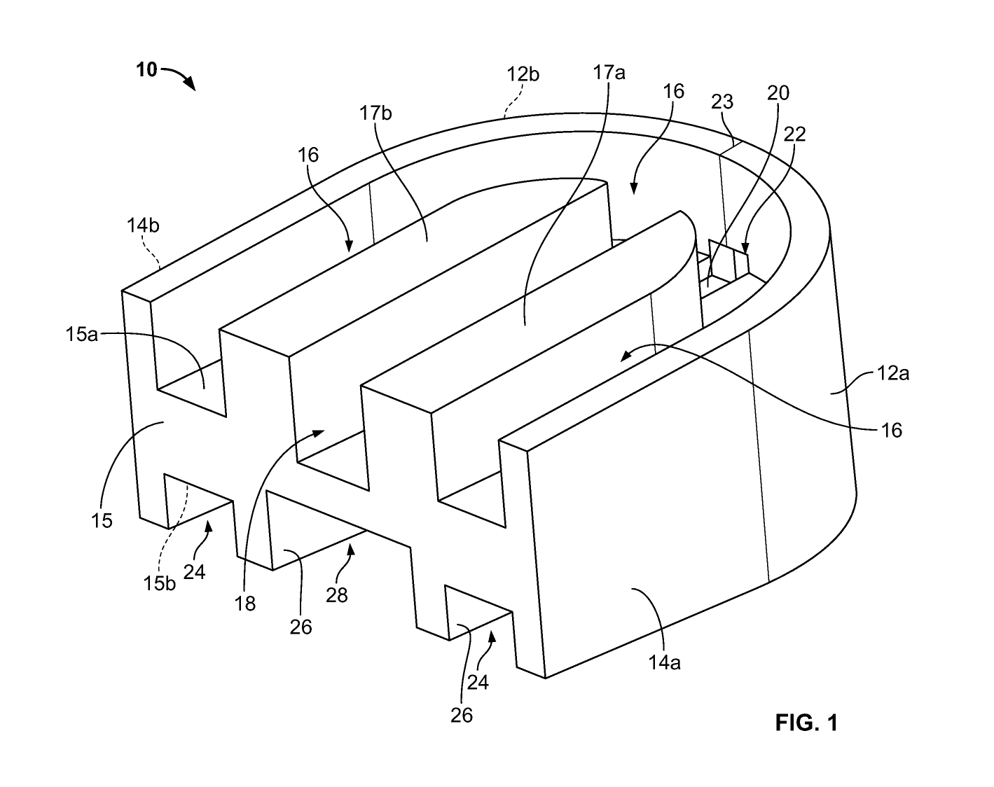

The present disclosure relates to a disinfecting mouth guard for VAP prevention, as discussed in detail below in connection with FIGS. 1-5.

FIG. 1 is a top perspective view of the mouth guard 10. The mouth guard 10 includes first and second curved walls 12a, 12b, first and second side walls 14a, 14b, and a central horizontal wall 15. The first side wall 14a, first curved wall 12a, second curved wall 12b, and second side wall 14b are connected such that they form an outer perimeter of the mouth guard 10. The first and second curved walls 12a, 12b can be integrally formed together, and/or with the side walls 14a, 14b. The apex formed by the first curved wall 12a and the second curved wall 12b corresponds to the distal end of the mouth guard, and the free ends of the first and second side walls 14a, 14b corresponds to the proximal end. The outer perimeter (formed by the first and second curved walls 12a, 12b, and first and second side walls 14a, 14b) is shaped to surround and guard a patient's teeth. The central wall 15 extends from the proximal end to the distal end and is connected with the first and second curved walls 12a, 12b and the first and second side walls 14a, 14b. The central wall 15 includes top and bottom surfaces 15a, 15b and can be positioned between a patient's upper and lower sets of teeth.

A top channel 16 for receiving the patient's upper teeth extends about the inner face of the first and second curved walls 12a, 12b, and the first and second side walls 14a, 14b, and is further defined by first and second protrusions 17a, 17b. The top channel 16 is generally horseshoe shaped. The first and second protrusions 17a, 17b extend from the top surface 15a of the central wall 15 and define a first tube channel 18 therebetween, for receiving an intubation tube. The first tube channel 18 extends into the central wall 15 such that there is depth between the top surface 15a and the bottom of the first tube channel 18. The first tube channel 18 extends from the proximal end of the mouth guard 10 toward the distal end, where it connects with a second tube channel 20. The second tube channel 20 is formed in the central wall 15 as a recess extending into the top surface 15a. The tube first channel 18 and the second tube channel 20 can have the same depth. The second tube channel 20 extends to the distal end of the mouth guard 10 where it connects with an opening 22. The opening 22 is positioned at the distal end of the mouth guard 10, and extends through the first and second curved walls 12a, 12b. Accordingly, the first tube channel 18, the second tube channel 20, and the opening 22 provide a pathway connecting the proximal end of the mouth guard 10 with the distal end of the mouth guard 10.

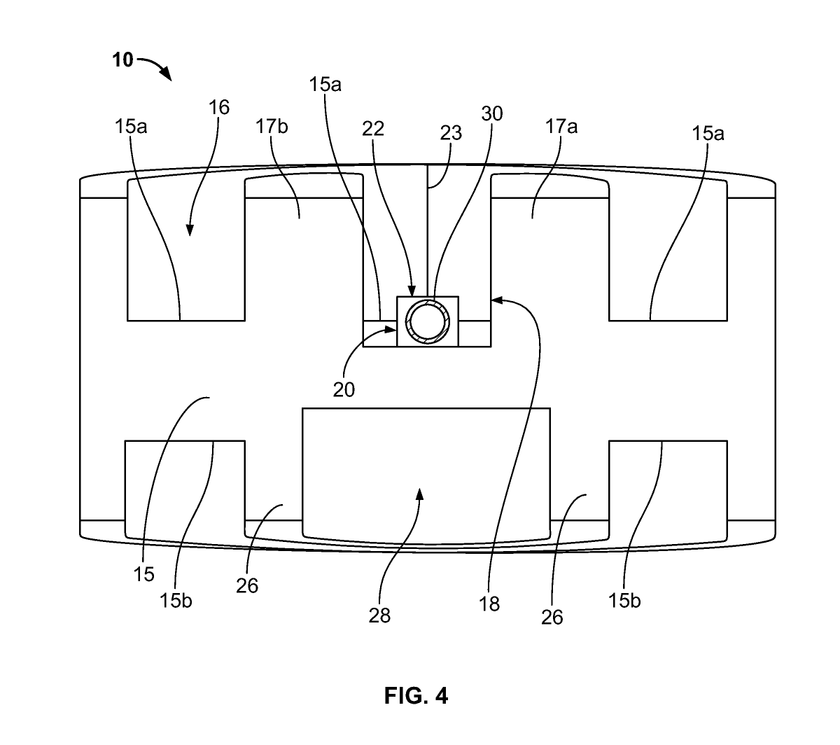

The arrangement of the first tube channel 18, the second tube channel 20, and the opening 22 allows a tube 30 (shown in FIG. 4) to be positioned through the opening 22 and extend across the first and second tube channels 18, 20 to the proximal end of the mouth guard 10, where it can extend into a patient's throat. A slit 23 is provided in the mouth guard 10 at the interface (apex) of the first and second curved walls 12a, 12b, and extends downwardly toward the opening 22. The slit 23 allows the tube 30 to slide down into the opening 22 and second tube channel 20, allowing the slit 23 to close and reform around the upper teeth. Additionally, placement of the tube 30 in the first and second tube channels 18, 20 prevents the patient's upper teeth from fully compressing the tube 30 because there is clearance between the top surface 15a of the central wall 15 and the bottoms of the first and second tube channels 18, 20. In other words, the bottom of the tube channels 18, 20 are lower than the top surface 15a.

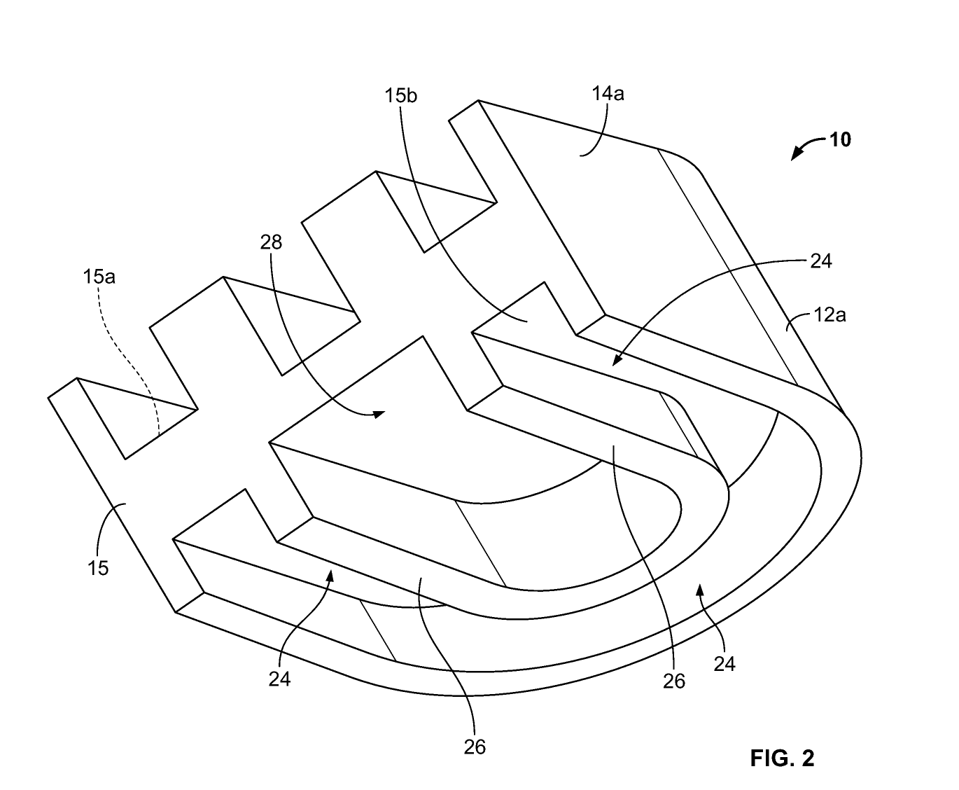

FIG. 2 is a bottom perspective view of the mouth guard 10. A bottom channel 24 extends about the inner face of the first and second curved walls 12a, 12b, and the first and second side walls 14a, 14b, and is further defined by a tongue guard 26. The bottom channel 24 is generally horseshoe shaped and receives the patient's bottom set of teeth. The tongue guard 26 is a generally horseshoe shaped protrusion that extends from the bottom surface 15b of the central wall 15 and defines a space 28 for receiving a patient's tongue. The space 28 can include a recessed portion that extends into the central wall 15 near the proximal end of the mouth guard 10 to provide the patient's tongue with additional space.

The mouth guard 10 need not be horseshoe in shape, but could have various shapes and sizes. For example, some instances may require only a portion of a patient's mouth and/or teeth to be disinfected. In such instances, the mouth guard 10 can be shaped to match only the portion of the patient's mouth and/or teeth which requires disinfecting. This can be, for example, the top set of teeth, the bottom set of teeth, the incisors, the cuspids and/or bicuspids, the molars, or any combination thereof. Similarly, the mouth guard 10 can be sized to cover the gum line. The mouth guard 10 could also be provided as two portions, i.e., upper and lower portions. Such an arrangement would allow the upper and lower mouth portions to move independent of one another.

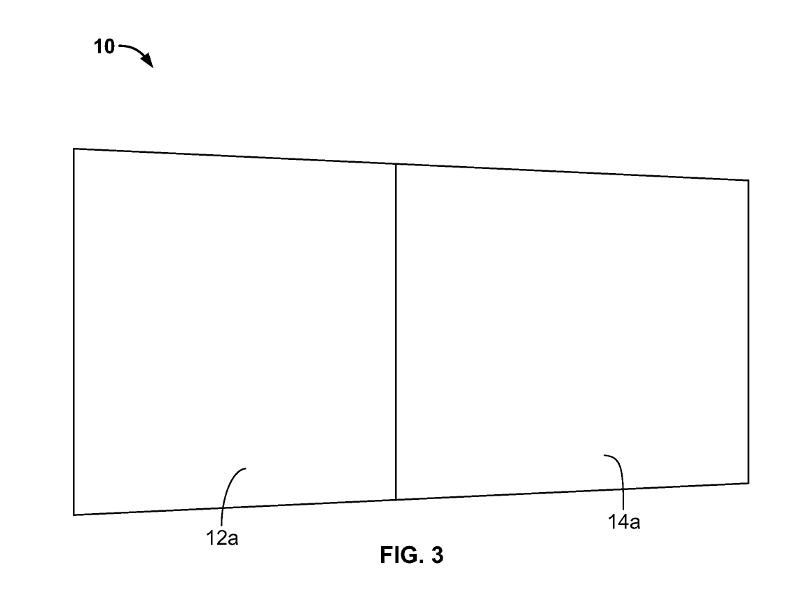

FIG. 3 is a right side view of the mouth guard 10. As can be seen, the mouth guard 10 can be tapered in shape. It should be understood that the left side view of the mouth guard 10 is a minor image of the right side view. As can be seen in FIG. 3, the mouth guard 10 decreases in height from the distal end, e.g., the end to be positioned toward the open mouth, to the proximal end, e.g., the end to be positioned closer to the throat. The tapered shape allows the mouth guard 10 to better conform to the shape of a patient's open mouth. Accordingly, one of ordinary skill in the art would understand that the taper can be any angle that is adequate and/or desirable, and can be customized for different patients.

Alternatively, the mouth guard 10 can have different shapes as necessitated by the patient. For example, instead of a taper, the mouth guard 10 can have a more complicated shape that conforms to the patient's mouth, or to specific portions thereof. Accordingly, the mouth guard 10 can be shaped so that it disinfects the necessary areas of a patient's mouth. FIG. 4 is a rear view of the mouth guard 10 showing the proximal end, which would be positioned near the throat of a patient. FIG. 4 shows the tube 30 positioned within the opening 22, the second tube channel 20, and the first tube channel 18. Accordingly, the tube 30 can extend through the mouth guard 10 and into a patient's throat. As can be seen in FIG. 4, there is clearance between the top surface 15a of the central wall 15 and the bottom of the first and second tube channels 18, 20 which prevents a patient from completely compressing the tube 30 with his or her teeth. The tube 30 can have varying diameters, and the clearance between the top surface 15a of the central wall 15 and the bottom of the first and second tube channels 18, 20 can also be of varying height.

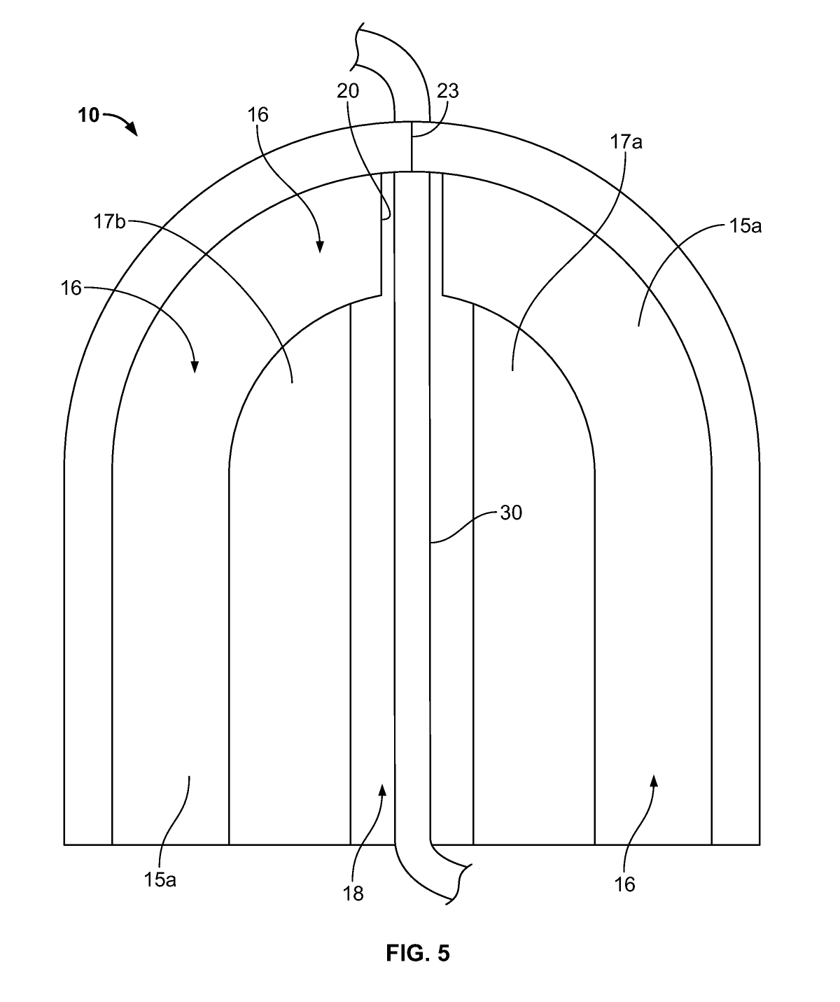

FIG. 5 is a top view of the mouth guard 10. As can be seen in FIG. 5, the first channel 18 and the second channel 20 are interconnected to create a path between the proximal and distal ends of the mouth guard 10, as previously described in detail. The tube 30 is seated in the chambers 18, 20, and the patient's upper teeth are positioned in the top channel 16. As discussed above, the mouth guard 10 can be formed as two separate pieces, e.g., a top and bottom mouth guard. In such a configuration, the top and bottom pieces of the mouth guard 10 could include an opening such that a pathway is formed between the two pieces when they are adjacent to one another, to permit passage of the tube 30 into the patient's mouth and throat.

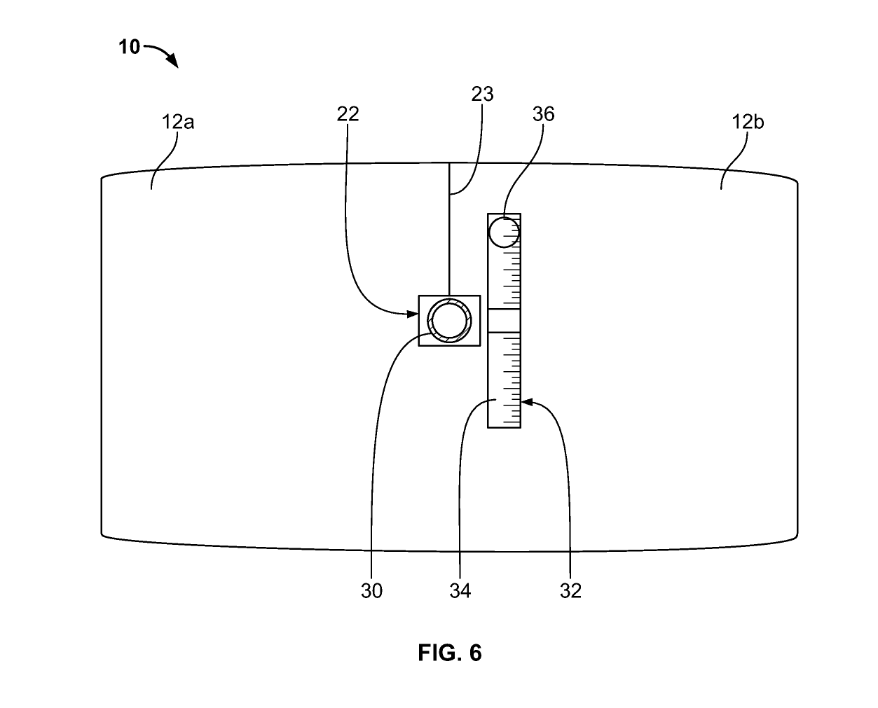

FIG. 6 is a front view of another embodiment of the mouth guard 10 which includes a level indicating device 32 that can indicate the angle of the patient's head (e.g., whether the head is positioned at an ideal angle of 30-45 degrees). The level indicating device 32 can be mounted to the first or second curved walls 12a, 12b, or to the first or second sidewalls 14a, 14b. The level indicating device 32 could be a spirit or bubble level that includes a tube 34 filled with a liquid, e.g., alcohol or water, and includes a bubble 36. The tube 34 can include markers and/or numerical indicia that allow an individual to quickly determine the angle of the patient's head. Of course, the level indicating device 32 could be any other device that is capable of indicating the tilt of the patient's head. For example, the level indicating device 32 can be a tilt meter, a weighted member (e.g., a plumb bob), or any other suitable device.

Further, the mouth guard 10 can be made of a sponge material, which can be any suitable sponge or sponge-like material including polyurethane, silicone, polyethylene, cotton, cellulose, regenerated cellulose, gauze, polyester, polyvinyl alcohol, non-woven materials, polypropylene, porous plastic, bonded fiber, latex, polyolefins, nylon, cellulosics, acetates, etc. Additionally, the mouth guard 10, can be pre-saturated with an anti-microbial, such as chlorhexidine gluconate, chlorhexidine acetate, cetylpyridinium chloride, hydrogen peroxide, ethanol, triclosan, sodium bicarbonate, menthol, thymol, methyl salicylate, and eucalyptol. The mouth guard 10 can also be embedded with other anti-microbial materials such as silver, chlorhexidine gluconate, chlorhexidine acetate, etc. In addition, antibiotics, such as rifampin or minocycline, could also be incorporated into the mouth guard 10 to provide an anti-bacterial effect. The mouth guard 10 is shaped to be placed in the oral cavity around the mouth, teeth, and/or gums, but still allow proper placement and positioning of the intubation tube 30. The mouth guard 10 can fill the entire oral cavity, or can be sized to fill only a portion of the oral cavity, e.g., to only cover certain teeth or a portion of the gums. That is, the mouth guard 10 can be strategically designed to only cover certain areas that are prone to infection.

Alternatively, the mouth guard 10 could be made of a soft polymer or rubber formulation that is embedded with an antimicrobial, such as those described above. This may avoid risks of aspiration or fluid dripping down into a patient's airway. Moreover, the mouth guard 10 could be made of a gel-like material that can be mold-able to the shape of a patient's mouth. This could provide more direct contact between the mouth guard 10 and the interior structure of the patient's mouth for improved anti-microbial performance. The gel materials can be any material that is capable of being molded, but resilient to deformation over time. Such gel materials could include hardening gels, soft polymers, or rubber materials.

The disinfectant contained within the mouthpiece could incorporate a residual effect that could provide continued anti-microbial properties over time even after the mouth guard 10 is removed. Examples of such disinfectants include chlorhexidine gluconate and chlorhexidine acetate. Further, the mouth guard 10 can be saturated with enough disinfectant solution so that it remains wetted throughout for the duration of use, but does not dispense enough solution upon application to induce respiratory issues with the patient.

The mouth guard 10 can be sterilized prior to use by patient. For example, the mouth guard 10 can be sterilized by various methods, including utilizing gamma radiation, ethylene oxide sterilization, moist heat, and other means.

The mouth guard 10 could be provided to a customer in a package, such as a foil laminate structure or other material suitable for preventing the mouth guard 10 from drying out in storage. Additionally, the mouth guard 10 can include an applicator for placing the sponge into a patient's mouth.

The mouth guard 10 could also perform other functions, such as oral lubrication to avoid drymouth, a common ailment suffered by those on ventilators.

Other features could be provided. For example, the mouth guard 10 could include a hinge along its length to facilitate fitting within the oral cavity of a patient. Additionally, the mouth guard 10 could include a suction port incorporated therein. The suction port could provide means for removal of any dispensed disinfectant solution, or other oral fluids that are desired to be removed from the oral cavity. Still further, the mouth guard 10 could be provided with flavoring, including mint, cinnamon, spearmint, menthol, artificial sweeteners, fruit or other desirable flavors that may please the patient, partially sedate the patient, and/or provide for patient comfort.

The mouth guard 10 could incorporate a re-use prevention feature. For example, a color changing additive could be incorporated into the base material of the mouth guard 10. The color changing additive could change color over a desired time period that would equate to an allowable use period for the product, after which its anti-microbial effects may lessen.

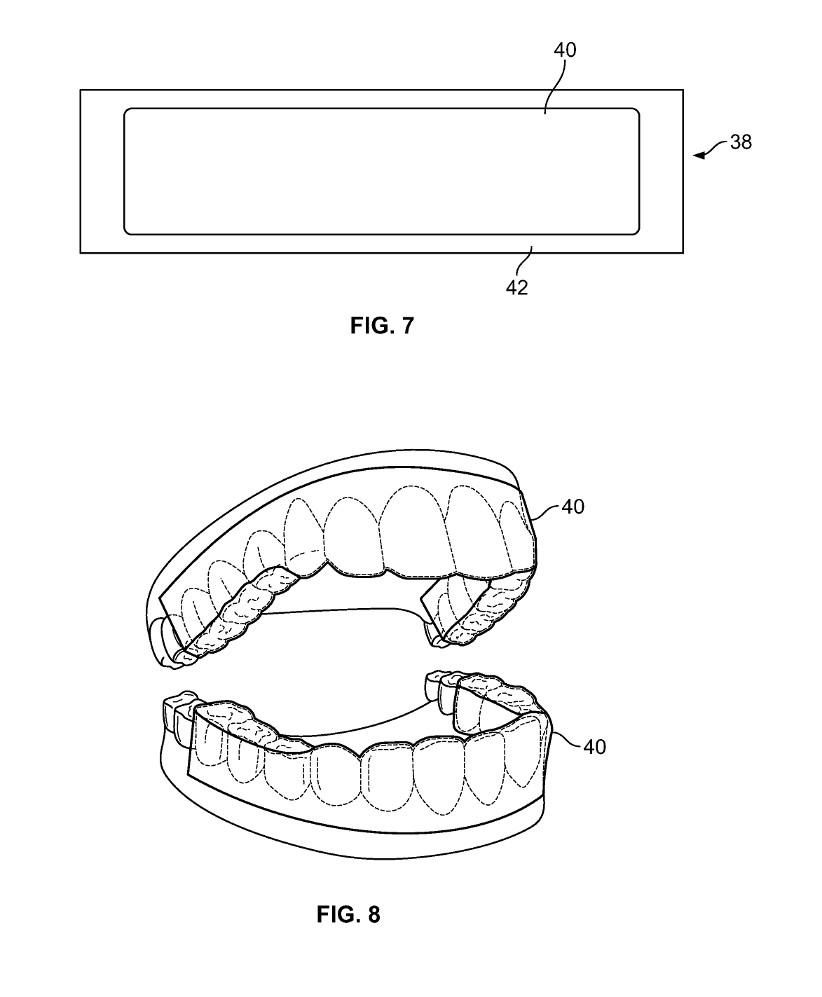

FIG. 7 is a front view of another embodiment of the mouth guard, indicated at 38, formed as an adhesive strip material. The mouth guard 38 could include a strip 40 disposed on a removable backing 42. The strip 40 could be a thin polyethylene film that could be applied to the interior surfaces of a patient's mouth (e.g., the teeth or gums). The strip 40 could be wetted with water and/or glycerin, and could include a pre-applied antimicrobial/disinfectant, such that the application of the strip to the patient could transfer the antimicrobial/disinfectant to the oral surfaces. Examples of such application include directly applying the antimicrobial/disinfectant in liquid, powder, or gel form to the strip. Example antimicrobials/disinfectants include chlorhexidine acetate, cetylpyridinium chloride, hydrogen peroxide, ethanol, triclosan, sodium bicarbonate, menthol, thymol, methyl salicylate, eucalyptol, silver, chlorhexidine gluconate, and chlorhexidine acetate.

FIG. 8 is a perspective view showing application of the strip 40 to a patient's mouth. As can be seen, the strip 40 can cover the patient's teeth and gums. Similar to the mouth guard 10, discussed above, the strip 40 can be of varying shapes and sizes, such that it can cover an entire set of teeth, only a few teeth, the gums, etc. A bite block could be used in conjunction with the strip 40 to prevent the patient from biting the intubation tube.

Prior to using the mouth guard 10, a nurse or other person could initially provide an oral scrub to a patient using a traditional foam swab tip or a traditional toothbrush available on the market today. This can remove loose plaque, food, or mouth debris from the teeth and gums. Upon completion of the oral scrub, the oral disinfecting mouth guard 10 can be placed into the patient's mouth and around the intubation tube 30 to act as further microbial barrier to any organisms that remain in the mouth, on the teeth, or even in the plaque on the teeth. The mouth guard 10 will also prevent any airborne bacteria from entering the mouth and further reducing the risk of infection.

Having thus described the disclosure in detail, it is to be understood that the foregoing description is not intended to limit the spirit or scope thereof. Accordingly, although the present disclosure has been described with reference to particular embodiments thereof, it is understood by one of ordinary skill in the art, upon a reading and understanding of the foregoing disclosure, that numerous variations and alterations to the disclosed embodiments will fall within the spirit and scope of the present disclosure and of the appended claims.

What is claimed is:

1. A mouth guard, comprising:
first and second side walls;
first and second curved walls connected to the first and second side walls, the side walls and the curved walls defining a periphery of the mouth guard;
a central wall having a top surface and a bottom surface, the central wall extending between the first and second sidewalls, and the first and second curved walls;
a top channel for receiving a patient's upper teeth;
a bottom channel for receiving a patient's lower teeth;
first and second protrusions extending from the top surface of the central wall;
a first tube channel formed between the first and second protrusions;
a second tube channel formed in the central wall and connected with the first tube channel; and
an opening formed in the first and second curved walls and in communication with the second tube channel,
wherein the first and second channels, and the opening receive an intubation tube.

2. The mouth guard of claim 1, further comprising a slit extending through the curved walls to the opening,
wherein the slit separates to allow a tube to slide into the opening, and closes when the tube is positioned in the opening.

3. The mouth guard of claim 1, further comprising a tongue guard extending from the bottom surface of the central wall, the tongue guard forming a space for receiving a patient's tongue.

4. The mouth guard of claim 1, wherein the mouth guard is formed of a sponge-like material.

5. The mouth guard of claim 4, wherein the sponge-like material is selected from the group of polyurethane, silicone, polyethylene, cotton, cellulose, regenerated cellulose, gauze, polyester, polyvinyl alcohol, non-woven materials, polypropylene, porous plastic, bonded fiber, latex, polyolefins, nylon, cellulosics, and acetates.

6. The mouth guard of claim 1, wherein the mouth guard is formed of a soft polymer or rubber formulation.

7. The mouth guard of claim 1, wherein the mouth guard is formed of gel-like material that is mold-able.

8. The mouth guard of claim 1, wherein the mouth guard is pre-saturated with an anti-microbial.

9. The mouth guard of claim 8, wherein the anti-microbial is selected from the group consisting of chlorhexidine gluconate, chlorhexidine acetate, cetylpyridinium chloride, hydrogen peroxide, ethanol, triclosan, sodium bicarbonate, menthol, thymol, methyl salicylate, and eucalyptol.

10. The mouth guard of claim 1, wherein the mouth guard is embedded with an anti-microbial material.

11. The mouth guard of claim 10, wherein the anti-microbial material is selected from the group consisting of silver, chlorhexidine gluconate, and chlorhexidine acetate.

12. The mouth guard of claim 1, further comprising a taper from a distal end of the mouth guard to a proximal end of the mouth guard.

13. The mouth guard of claim 1, wherein the mouth guard includes a disinfectant that provides continued anti-microbial action after the mouth guard has been removed from a patient's mouth.

14. The mouth guard of claim 13, wherein the disinfectant is one of chlorhexidine gluconate or chlorhexidine acetate.

15. The mouth guard of claim 1, further comprising a package that seals the mouth guard from external forces and prevents the mouth guard from drying out in storage.

16. The mouth guard of claim 1, further comprising an applicator for facilitating placement of the mouth guard into a patient's mouth.

17. The mouth guard of claim 1, further comprising a suction port for removing excess fluid.

18. The mouth guard of claim 1, further comprising a flavoring.

19. The mouth guard of claim 1, further comprising a color changing additive that changes color over a pre-determined period of time to signal the end of the useful life of the mouth guard.

20. The mouth guard of claim 1, further comprising a level indicating device, said level indicating device indicating an angle of a patient's head.

21. A mouth guard, comprising:
a body shaped to cover at least a portion of a patient's mouth, and
an antimicrobial substance,
wherein the antimicrobial substance prevents infections associated with ventilation of a patient.

22. The mouth guard of claim 21, wherein the body is formed from an absorbent material

23. The mouth guard of claim 22, wherein the absorbent material is a sponge material.

24. The mouth guard of claim 22, wherein the absorbent material is selected from the group of polyurethane, silicone, polyethylene, cotton, cellulose, regenerated cellulose, gauze, polyester, polyvinyl alcohol, non-woven materials, polypropylene, porous plastic, bonded fiber, latex, polyolefins, nylon, cellulosics, and acetates.

25. The mouth guard of claim 22, wherein the absorbent material is a soft polymer or rubber formulation.

26. The mouth guard of claim 22, wherein the absorbent material is pre-saturated with the antimicrobial substance.

27. The mouth guard of claim 21, wherein the body is formed of a moldable, gel-like material.

28. The mouth guard of claim 21, wherein the antimicrobial substance is selected from the group consisting of silver, chlorhexidine gluconate, chlorhexidine acetate, cetylpyridinium chloride, hydrogen peroxide, ethanol, triclosan, sodium bicarbonate, menthol, thymol, methyl salicylate, and eucalyptol.

29. The mouth guard of claim 22, wherein the absorbent material is embedded with the antimicrobial substance.

30. The mouth guard of claim 21, wherein the body includes a channel for receiving at least one of a patient's teeth.

31. A mouth guard, comprising:
a strip including an antimicrobial substance suitable for preventing infections associated with ventilation of a patient; and
means for adhering the strip to a patient's mouth.

32. The mouth guard of claim 31, wherein the strip is a polyethylene film.

33. The mouth guard of claim 31, wherein the antimicrobial substance is pre-applied to the strip.

34. The mouth guard of claim 31, wherein the antimicrobial substance is selected from the group consisting of chlorhexidine gluconate, chlorhexidine acetate, cetylpyridinium chloride, hydrogen peroxide, ethanol, triclosan, sodium bicarbonate, menthol, thymol, methyl salicylate, and eucalyptol.

35. The mouth guard of claim 31, wherein the strip is embedded with the antimicrobial substance.

36. A method of preventing ventilator associated pneumonia, comprising:
providing a mouth guard having an antimicrobial substance; and
positioning the mouth guard in a patient's mouth,
wherein said antimicrobial substance disinfects at least a portion of the patient's mouth to prevent infection.
